(19) United States
(12) Patent Application Publication
(10) Pub. No.: US 2003/0216702 A1
(43) Pub. Date: Nov. 20, 2003

(54) Ostomy waste disposal kit

(76) Inventor: Gary Lynn Butler, Yazoo City, MS (US)

Correspondence Address: Gary Butler, 1520 Park Ave, Yazoo City, MS, 39194, US

(21) Appl. No.: 10/081,033

(22) Filed: May 20, 2002

(51) Int. Cl. A61F 5/44

(52) U.S. Cl. 604/332

(57) ABSTRACT

I, Gary Butler, summarize the invention as follows: the OSTOMY WASTE DISPOSAL KIT will enable ostomy patients to dispose of solid waste in a bucket at any location. It benefits patients in medical and nursing institutions, even on trips. By placing the bucket's safety chain around the patient's neck where the bucket is suspended under the ostomy pouch and the stool is deposited and the pouch then cleansed with the water in the squirt bottle, the kit could be utilized at any restroom at any location.

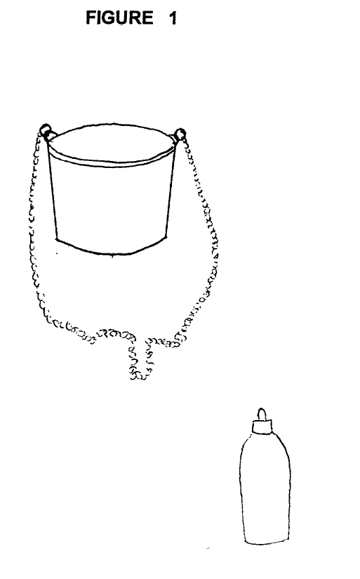

One 8 quart steel bucket, one small chain 5 to 6 feet in length, two regular steel key rings that are placed thru both ends of the chain and then thru the handle support ring on each side of the buckets enabling the bucket to be adjusted in proportion to the patient's height.

When in use, the patient or caregiver places the chain around the patients neck, allowing the bucket to be suspended directly underneath the ostomy pouch. The pouch clamp is then removed to allow the stool residue to be deposited in the bucket. Water in the squirt bottle is then applied to the internal portion of the pouch for final cleansing before re-clamping the pouch. The chain is then removed from the patient's neck and the stool residue can be rinsed from the bucket and deposited in a toilet.

The kit would be very beneficial to all ostomy patients in medical and nursing institutions, homes, and if placed in a utility bag, on trips.

A color photograph of the ostomy waste disposal kit is provided for your review.

I, Gary Butler, claim my invention will enable ostomy patients ( persons who must wear ostomy pouches) to dispose of solid waste (stool) collected in the pouches with convienance at any location.

1. Parts of the invention include a 12 ounce squirt bottle, an 8 quart steel bucket, one small safety chain 5 feet in length, two ½ inch regular steel key rings that are placed thru both ends of the chain and then thru the handle support ring on each side of the bucket, enabling the bucket to be adjusted in proportion to the patient's height.

2. When in use, the patient or caregiver places the chain around the patient's neck, allowing the bucket to be suspended directly underneath the ostomy pouch. The pouch clamp is then removed to allow the stool residue to be deposited in the bucket. Water in the squirt bottle is then applied to the internal portion of the pouch for final cleansing before re-clamping the pouch. The chain is then removed from the patients neck and the stool residue can be rinsed from the bucket and deposited in a toilet.

3. The kit would be beneficial to all ostomy patients in medical and nursing institutions, homes, and, if placed in a utility bag, on trips.
